(19) United States
(12) Patent Application Publication
(10) Pub. No.: US 2023/0095701 A1
(43) Pub. Date: Mar. 30, 2023

(54) Use of Albiflorin in Treatment of Coronavirus Pneumonia

(71) Applicant: Zuoguang Zhang, Beijing (CN)

(72) Inventors: Zuoguang Zhang, Beijing (CN); Hui Tian, Beijing (CN)

(21) Appl. No.: 17/802,806

(22) PCT Filed: Sep. 25, 2020
(86) PCT No.: PCT/CN2020/117638

§ 371 Date: Aug. 26, 2022

(30) Foreign Application Priority Data
Feb. 27, 2020 (CN) 202010126371X

(51) Int. Cl. A61K 31/7048; A61K 36/65; A61P 29/00

(52) U.S. Cl. CPC A61K 31/7048; A61K 36/65; A61P 29/00

(57) ABSTRACT

Use of albiflorin or a pharmaceutically acceptable salt thereof or an extract or a pharmaceutical composition containing albiflorin in the preparation of a medicament for preventing or treating coronavirus pneumonia, especially novel coronavirus pneumonia, or use thereof in the preparation of a medicament for treating prolonged symptoms of novel coronavirus pneumonia, performing rehabilitation conditioning after recovery of novel coronavirus pneumonia, or alleviating a possible sequela of novel coronavirus pneumonia, the sequela being depression, anxiety, sleep disorder, pain, palpitation, asthma, intestinal function disorder, or chronic fatigue syndrome. Albiflorin has the effects of anti-coronavirus, anti-inflammatory, inflammatory storm inhibition, human microecological balance regulation, etc., can comprehensively prevent and treat coronavirus-induced pneumonia, and can be prepared into medicaments, health care products or nutrition regulators for preventing or treating coronavirus pneumonia, especially novel coronavirus pneumonia.

### TECHNICAL FIELD

The present invention belongs to the field of medicines. Specifically, the present invention relates to albiflorin or a pharmaceutically acceptable salt thereof or an extract or a pharmaceutical composition containing albiflorin in the treatment of coronavirus pneumonia, in particular novel coronavirus pneumonia (COVID-19). The present invention further relates to a medicament, a health care product or a nutrition regulator for preventing or treating coronavirus pneumonia, in particular novel coronavirus pneumonia, including albiflorin or a pharmaceutically acceptable salt thereof or an extract or a pharmaceutical composition containing albiflorin.

### BACKGROUND ART

Since December 2019, multiple pneumonia cases of unknown cause have been successively found in Wuhan City, Hubei Province. It has been studied that this pneumonia is an acute respiratory infectious disease caused by novel coronavirus (2019-nCoV) of β genus. Upon genetic sequencing, 2019-nCoV has been found to be highly homologous to known coronaviruses SARS-CoV and MERS-CoV, which have the almost same process of invading hosts with 2019-nCoV, so corresponding drug development strategies should be roughly the same. A pathological process that affects the entry of coronavirus into the host and the use of cells to invade and reproduce to generate new viruses mainly relies on four key proteases, Spike, 3CLpro, RdRp and PLpro, which, therefore, have become the most important targets for the development of anti-coronavirus drugs.

At present, the complete gene sequencing of 2019-nCoV has been completed, and found:

1. Compared with a Spike protein of SARS-CoV, a Spike protein of 2019-nCoV has undergone major changes in some key areas, resulting in a decline in the therapeutic efficacy of drugs targeting the Spike protein of SARS-CoV for 2019-nCoV. The University of Texas at Austin and the National Institutes of Health of the United States have reconstructed 2019-nCoV using a cryo-electron microscopy based on a viral gene sequence provided by Chinese researchers, and found that the Spike protein of 2019-nCoV is similar in structure to that of SARS-CoV, but has an affinity for ACE2 is 10-20 times that of SARS-CoV. Their researches have proved once again that previous drugs targeting the Spike protein may not have an obvious inhibitory effect on the novel coronavirus.

2. Correspondingly, the three key targets of 3CLpro, RdRp and PLpro of 2019-nCoV have more than 95% sequence similarity with those of SARS-CoV. Therefore, some researchers have proposed that active compounds developed for the three biological targets of SARS-CoV, 3CLpro, RdRp and PLpro, may have a relatively obvious curative effect on COVID-19.

However, apart from the new use of old drugs such as chloroquine phosphate (for treatment of malaria) and arbidol (for treatment of influenza caused by influenza A and B viruses), which have been included in the sixth edition of the Diagnosis and Treatment Plan, there have been no reports or disclosures of antiviral drugs developed against these three biological targets for the treatment of COVID-19.

Bile acid (BA) plays an important role in the immune response of human cells to external viruses. The antiviral ability can be reduced to varying degrees if a signal flow of bile acid in the body is blocked, suggesting that the bile acid has a clinical antiviral potential. The BioRxiv website recently disclosed that Italian scientists had found that bile acid in the human body can prevent 2019-nCoV from attacking healthy cells: “it starts to work when the number of viruses (invading the human body) is not very high, so as to prevent infection”. This finding provides a therapeutic approach for taking different measures to prevent 2019-nCoV invasion. As disclosed by the director of this project, Professor Angela Zampella, Head of the Faculty of Pharmacy at the University of Naples Federico II, this antiviral study is the first step in formulating a treatment regimen for COVID-19, “which will be submitted to the Agenzia Italiana del Farmaco (AIFA) for review and reference”. Regarding an immune relationship between the gut and the lung, an important study was published in the journal “Science” in 2018, which mentioned: “natural lymphocytes involved in the body's pathological processes such as homeostasis, asthma and chronic obstructive pulmonary disease etc., migrate from the gut to the lung to participate in the pulmonary immune response”. Pulmonary-enteric circulation is at present a hotspot in medical researches of acute and critical cases, and the bile acid can escape into the lung through the pulmonary-enteric circulation. Applicants' collaborators at UCSD Medical Center detected the bile acid in bronchoalveolar lavage fluid (BALF) of rat lungs using a targeted metabolomic approach. The results of this study support Italian scientists' claim that the bile acid can prevent 2019-nCoV from attacking healthy cells in the lungs.

In addition to antiviral treatment, in this epidemic, medical staff found that anti-inflammation was very important in the treatment of COVID-19. Some patients are not very dangerous in the early stage, or even have mild symptoms, but in the later stage, the disease will suddenly worsen, and soon enter a state of “inflammatory storm”, resulting in a rapid failure of multiple organ functions, and ultimately threatening the life. The “inflammatory storm” is the culprit behind the death of many critically ill patients with COVID-19. Therefore, suppressing the “inflammatory storm” through effective anti-inflammation is a key to the treatment of COVID-19. Professor Kang Jingxuan of Harvard University, who has been nominated for the Nobel Prize in Biomedicine twice, said in an exclusive interview with 21st Century New Health: whether now or in the future, controlling inflammations plays an important role in reducing the COVID-19 mortality and promoting the faster recovery of patients. Professor Kang Jingxuan pointed out that the traditional Chinese medicine has played a great role in the anti-epidemic process, wherein a large part of the reasons is that the traditional Chinese medicine has achieved “anti-inflammation” in the treatment. Unfortunately, it has not been systematically rolled out, while the Western medicine has no feasible anti-inflammatory scheme. To this end, he suggested that in the fight against COVID-19, in addition to anti-virus, systemic treatment of inflammation must be strengthened, and anti-inflammation should be taken as part of the basic diagnosis and treatment plan for COVID-19. Regarding the use of hormones to fight inflammation, Professor Kang Jingxuan pointed out that “for example, with some hormones, we also want to control the inflammatory response, but there are many potential problems with high-dose or long-term use of hormones”. To this end, he suggested clinically integrating Chinese and Western anti-inflammatory drugs, evaluating them from each link and each pathway, and building a scientific, safe and effective anti-inflammatory scheme.

After the outbreak of the epidemic, major achievements have been made in the research on resisting inflammations and suppressing the “inflammatory storm” in China. As disclosed by the academician Zhou Qi, deputy secretary-general of the Chinese Academy of Sciences, researchers are screening some old drugs to suppress the “inflammatory storm”, including some anti-inflammatory drugs that have been proven effective in rheumatism and other fields.

The currently disclosed drugs for suppressing the “inflammatory storm” fall into the following categories:

1. Interleukin-6 Inhibitors

The team of Professor Wei Haiming from the University of Science and Technology of China, through a comprehensive analysis of 30 immunological indicators in the blood of 33 patients with COVID-19, found that interleukin 6 (IL-6) is an important pathway that induces the “inflammatory storm” of the novel coronavirus pneumonia, and an IL-6 inhibitor tocilizumab (ACTEMRA) was successfully used to block the “inflammatory storm”. In the “Notice on Printing and Distributing the Diagnosis and Treatment Plan for Severe and Critical Cases of Novel Coronavirus Pneumonia (Trial Version 2)” by the National Health Commission and the State Administration of Traditional Chinese Medicine, progressive elevation of IL-6 has been used as a clinical warning indicator of disease deterioration. Tocilizumab has been recommended by WHO for the treatment of COVID-19 worldwide.

2. Glycyrrhizic Acid Drugs

The clinical study on the combination of an anti-inflammatory drug diammonium glycyrrhizinate and vitamin C to inhibit inflammatory storm in the treatment of COVID-19 has been approved. The anti-inflammatory mechanism of the diammonium glycyrrhizinate is as follows: the diammonium glycyrrhizinate has a cortical hormone-like anti-inflammatory effect and can suppress in the initial stage the metabolic levels of phospholipase A2/arachidonic acid (PLA2/AA), NF-kB and MAPK/AP-1, the key inflammatory response signals induced by inflammatory stimuli, inhibit the activity of inflammatory response signals related to three inflammatory pathways, down-regulate the expression of related pro-inflammatory cytokines TNF-α, IL-8, IL-1β, IL-6, related chemokines and cyclooxygenase (COX) in the upstream of the inflammatory pathway, and block the generation of nitric oxide (NO), prostaglandin (PG), and reactive oxygen species (ROS) in the downstream inflammatory pathways.

3. Sphingosine Drugs

Using a sphingosine-1-phosphate (S1P) receptor signaling pathway, the immune pathological damage caused by the host's innate and adaptive immune responses can be reduced significantly, thereby reducing the morbidity and mortality of influenza virus infection. In addition, the treatment with S1P drugs can also generate certain anti-influenza virus, anti-SARS-CoV virus and anti-2019-nCoV T cells and antibodies to control infections. If the S1P drugs are used in combination with other antiviral drugs, the efficacy will be significantly improved.

According to the “Science and Technology Daily” report, a drug Opaganib developed by an Israeli pharmaceutical company can effectively treat the deadly disease caused by the novel coronavirus. This drug targets a unique enzyme called “sphingosine kinase 2 (SphK2)” and inhibits its activity. SphK2 is a component necessary for the replication of the novel coronavirus in cells. If it is inhibited, the virus will not replicate. Therefore, inhibiting SphK2 can both reduce inflammation levels and prevent viral replication. This drug has been approved by the Mexican Federal Commission for the Prevention of Health Risks and prepared to conduct a phase II/III study to evaluate the efficacy of the drug in patients with the novel coronavirus. In addition to Mexico, this drug is also approved in the UK and Russia. In addition, it is also being examined in Italy, Brazil and other countries.

4. IDO Inhibitors of Tryptophan Metabolic Pathway

The high expression of IDO1 leads to local depletion of tryptophan in cells, induces T cells to stagnate in G1 phase, inhibits the proliferation of T cells, and reduces the body's antiviral immune function. After mice are infected with influenza virus A/PR/8/34 (PR8), IDO1 activity is rapidly increased in the lung and in the mediastinal lymph nodes leading to the lung, resulting in increased lung inflammation, slowed convalescence, and reduced effector T cell responses. IDO1 inhibitors are used to fight viruses and reduce lung inflammations by improving the body's own immune function.

In the treatment of COVID-19, in addition to anti-virus and anti-inflammation, there is also an important treatment method, which is to reduce the secondary infection caused by bacterial translocation by improving the intestinal microecological balance of patients. The “Four-resistance Dual-balance” treatment model created by the Zhejiang team of Academician Li Lanjuan, director of the National Key Laboratory of Diagnosis and Treatment of Infectious Diseases, has played a major role in the treatment of critically ill patients with COVID-19 in the fight against the epidemic, and has been written into the “Diagnosis and Treatment Plan for Novel Coronavirus Infected Pneumonia (Trial Second Edition)”. The dual-balance in this model includes: 1, maintaining the acid-base balance of water-electrolyte; and 2, regulating the microecological balance of the human body.

In the treatment of COVID-19, doctors and researchers found that patients and recovered patients experienced prolonged symptoms. Even if the patients change from positive to negative after nucleic acid detection and are cured and discharged, many of them still experience symptoms such as pain, palpitation, asthma, consciousness disorder and chronic fatigue, etc. To this end, Mount Sinai Hospital in the United States has established a “Post-COVID-19 Care Center”, which has developed a physiotherapy rehabilitation plan for patients with autonomic dysfunction and symptoms of COVID-19. As disclosed by the “Wall Street Journal”, the proportion of patients with prolonged symptoms is 5-15%, which account for a huge group of patients. Taking the United States as an example, it currently exceeds 600,000. Meanwhile, an Italian study published in the Journal of the American Medical Association (JAMA) found that more than half of patients who recovered from COVID-19 continued to experience multiple symptoms for weeks after discharge, in which two of the symptoms were particularly pronounced: “one symptom is that a patient is feeling tired or extremely tired; and the other symptom is shortness of breath. The average duration of their symptoms was five weeks or more”. For patients with prolonged symptoms and patients with sequelae during the convalescence period after healing, for safety reasons, it is obvious that they cannot be treated with antiviral drugs (such as remdesivir, etc.) and anti-inflammatory biological preparations (such as Tocilizumab, etc.) any longer. Currently, there is no safe and appropriate scheme to the prolonged symptoms and sequelae of COVID-19.

In the fight against the epidemic, there is an urgent need to develop drugs for the treatment of COVID-19. In addition to the properties of resisting 2019-nCoV and inflammations and regulating the microecological balance of the human body, this drug should have certain anticoagulant, anti-hypoxia, anti-fatigue, anti-depression and anxiety effects, and also should have safety and few side effects, and ensure that patients can take this drug for a long time for conditioning treatment. In short, these drugs can maintain the health of patients during the whole course from prevention to treatment, and then to rehabilitation after recovery.

### SUMMARY OF THE INVENTION

An objective of the present invention is to meet the needs of prevention and treatment of coronavirus pneumonia (especially COVID-19), and to provide a new choice that can be used for the prevention and treatment of coronavirus pneumonia (especially COVID-19), and can be used for performing conditioning treatment during the convalescence period after healing. Specifically, the present invention provides use of albiflorin or a pharmaceutically acceptable salt thereof or an extract or a pharmaceutical composition containing albiflorin in the prevention, treatment or conditioning treatment during the convalescence period after healing. They can be prepared into medicaments for the prevention and treatment of influenza pneumonia and coronavirus pneumonia (especially COVID-19), and can also be prepared into health care products or nutritional regulators for the recovery of patients with COVID-19 after healing.

Albiflorin is a monoterpenoid compound with a molecular formula of C₂₃H₂₈O₁₁ and a molecular weight of 480.46, and has a molecular structure shown in the following formula (I). It is a natural active substance derived from Paeonia lactiflora Pall or the root of Paeonia veitchii Lynch, and the root of P. suffrsticosa Andrz.

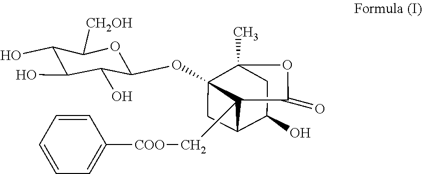

In the context of the present invention, the term “an extract or a pharmaceutical composition containing albiflorin” refers to any extract or pharmaceutical composition containing albiflorin. For example, the extract containing albiflorin can be total glucosides of Paeonia lactiflora Pall and/or a Paeonia lactiflora Pall extract, and the pharmaceutical composition containing albiflorin can be a Chinese herbaceous peony and licorice preparation, wherein the Chinese herbaceous peony and licorice preparation is a preparation prepared from the following raw materials: medicinal materials of Chinese herbaceous peony and licorice, and/or a Chinese herbaceous peony extract and a licorice extract.

It is found upon the inventor's researches that albiflorin has the following effects:

1. 2019-nCoV is resisted by inhibiting a 3CLpro protein and promoting human endogenous bile acid secretion;

2. an anti-inflammatory effect is achieved by inhibiting inflammatory factors such as interleukin-6, phospholipase A2/arachidonic acid (PLA2/AA), inhibiting SphK1 and SphK2, and inhibiting inflammatory pathways such as IDO1, thereby preventing “inflammatory storm”;

3. secondary infection caused by bacterial translocation is inhibited by improving the microecological balance of the human body;

4. by increasing the level of erythropoietin (EPO) under the symptoms of body hypoxia, erythropoiesis is promoted to increase the oxygen-carrying capacity of hemoglobin, and alleviate the lack of blood oxygen saturation caused by coronavirus pneumonia, especially novel coronavirus pneumonia; and

5. by regulating the intestinal flora balance, promoting the production of endocannabinoids and resisting hypoxia and fatigue, a conditioning treatment for patients with novel coronavirus pneumonia is performed on the prolonged symptoms, the sequelae after healing and the recovery of physical strength during the rehabilitation period.

Therefore, the objective of the present invention is achieved by the following technical solutions:

In one aspect, the present invention provide use of albiflorin or a pharmaceutically acceptable salt thereof or an extract or a pharmaceutical composition containing albiflorin in the preparation of a medicament for preventing or treating coronavirus pneumonia, wherein the coronavirus pneumonia is preferably novel coronavirus pneumonia (COVID-19); more preferably, the albiflorin or the pharmaceutically acceptable salt thereof or the extract or pharmaceutical composition containing albiflorin is used to prevent or treat the coronavirus pneumonia by one or more of the following ways: resisting coronavirus, resisting inflammatory storm, restoring intestinal flora balance and resisting hypoxia; and further preferably, the albiflorin or the pharmaceutically acceptable salt thereof or the extract or pharmaceutical composition containing albiflorin is used to prevent or treat the coronavirus pneumonia by one or more of the following ways: inhibiting coronavirus 3CLpro protein, promoting endogenous bile acid secretion, inhibiting sphingosine kinase SphK1 and/or SphK2, inhibiting interleukin-6 or phospholipase A2/arachidonic acid (PLA2/AA) inflammatory factors, inhibiting an IDO1 inflammatory signaling pathway, regulating intestinal flora balance and promoting EPO production to resist hypoxia.

In another aspect, the present invention provides use of albiflorin or a pharmaceutically acceptable salt thereof or an extract or a pharmaceutical composition containing albiflorin in the preparation of a medicament for treating prolonged symptoms of novel coronavirus pneumonia, performing rehabilitation conditioning after recovery of novel coronavirus pneumonia, or alleviating a possible sequela of novel coronavirus pneumonia, wherein the prolonged symptoms of novel coronavirus pneumonia are pain, palpitation, asthma, consciousness disorder, or chronic fatigue; and/or the sequela is depression, anxiety, sleep disorder, pain, palpitation, asthma, intestinal function disorder or chronic fatigue syndrome, etc.

In certain embodiments of the present invention, the extract containing albiflorin is total glucosides of Paeonia lactiflora Pall and/or a Paeonia lactiflora Pall extract, and/or the pharmaceutical composition is a Chinese herbaceous peony and licorice preparation.

In yet another aspect, the present invention provides albiflorin or a pharmaceutically acceptable salt thereof or an extract or a pharmaceutical composition containing albiflorin for use in the prevention or treatment of coronavirus pneumonia, wherein the coronavirus pneumonia is preferably novel coronavirus pneumonia (COVID-19); more preferably, the albiflorin or the pharmaceutically acceptable salt thereof or the extract or pharmaceutical composition containing albiflorin is used to prevent or treat the coronavirus pneumonia by one or more of the following ways: resisting coronavirus, resisting inflammatory storm, restoring intestinal flora balance, and resisting blood coagulation and hypoxia; and further preferably, the albiflorin or the pharmaceutically acceptable salt thereof or the extract or pharmaceutical composition containing albiflorin is used to prevent or treat the coronavirus pneumonia by one or more of the following ways: inhibiting coronavirus 3CLpro protein, promoting endogenous bile acid secretion, inhibiting sphingosine kinase SphK1 and/or SphK2, inhibiting interleukin-6 or phospholipase A2/arachidonic acid (PLA2/AA) inflammatory factors, inhibiting an IDO1 inflammatory signaling pathway, regulating intestinal flora balance and promoting EPO production to resist hypoxia.

In another aspect, the present invention provides albiflorin or a pharmaceutically acceptable salt thereof or an extract or a pharmaceutical composition containing albiflorin for use in treating prolonged symptoms of novel coronavirus pneumonia, performing rehabilitation conditioning after recovery of novel coronavirus pneumonia, or alleviating a possible sequela of novel coronavirus pneumonia, wherein the prolonged symptoms of novel coronavirus pneumonia are pain, palpitation, asthma, consciousness disorder, or chronic fatigue; and/or the sequela is depression, anxiety, sleep disorder, pain, palpitation, asthma, intestinal function disorder or chronic fatigue syndrome, etc.

In certain embodiments of the present invention, in the albiflorin or the pharmaceutically acceptable salt thereof or the extract or pharmaceutical composition containing albiflorin, the extract containing albiflorin is total glucosides of Paeonia lactiflora Pall and/or a Paeonia lactiflora Pall extract, and/or the pharmaceutical composition is a Chinese herbaceous peony and licorice preparation.

In another aspect, the present invention provides a method for preventing or treating coronavirus pneumonia, comprising: administering a prophylactically or therapeutically effective amount of albiflorin or a pharmaceutically acceptable salt thereof or an extract or a pharmaceutical composition containing albiflorin to a subject in need thereof, wherein the coronavirus pneumonia is preferably novel coronavirus pneumonia (COVID-19); more preferably, the albiflorin or the pharmaceutically acceptable salt thereof or the extract or pharmaceutical composition containing albiflorin is used to prevent or treat the coronavirus pneumonia by one or more of the following ways: resisting coronavirus, resisting inflammatory storm, restoring intestinal flora balance and resisting hypoxia; and further preferably, the albiflorin or the pharmaceutically acceptable salt thereof or the extract or pharmaceutical composition containing albiflorin is used to prevent or treat the coronavirus pneumonia by one or more of the following ways: inhibiting coronavirus 3CLpro protein, promoting endogenous bile acid secretion, inhibiting sphingosine kinase SphK1 and/or SphK2, inhibiting interleukin-6 or phospholipase A2/arachidonic acid (PLA2/AA) inflammatory factors, inhibiting an IDO1 inflammatory signaling pathway, regulating intestinal flora balance and promoting EPO production to resist hypoxia.

In another aspect, the present invention provides a method for treating prolonged symptoms of novel coronavirus pneumonia, performing rehabilitation conditioning after recovery of novel coronavirus pneumonia, or alleviating a possible sequela of novel coronavirus pneumonia, comprising: administering a prophylactically or therapeutically effective amount of albiflorin or a pharmaceutically acceptable salt thereof or an extract or a pharmaceutical composition containing albiflorin to a subject in need thereof, wherein the prolonged symptoms of the novel coronavirus pneumonia are pain, palpitation, asthma, consciousness disorder, or chronic fatigue; and/or the sequela is depression, anxiety, sleep disorder, pain, palpitation, asthma, intestinal function disorder or chronic fatigue syndrome, etc.

In certain embodiments of the present invention, the extract containing albiflorin is total glucosides of Paeonia lactiflora Pall and/or a Paeonia lactiflora Pall extract, and/or the pharmaceutical composition is a Chinese herbaceous peony and licorice preparation.

In another aspect, the present invention provides a medicament, a health care product or a nutrition regulator for preventing or treating coronavirus pneumonia, comprising albiflorin or a pharmaceutically acceptable salt thereof or an extract or a pharmaceutical composition containing albiflorin, wherein the extract containing albiflorin is preferably total glucosides of Paeonia lactiflora Pall and/or a Paeonia lactiflora Pall extract, and/or the pharmaceutical composition is a Chinese herbaceous peony and licorice preparation; more preferably, the medicament, health care product or nutrition regulator is selected from capsules, tablets, dropping pills, preparations for nasal administration or injections, etc; more preferably, the albiflorin or the pharmaceutically acceptable salt thereof or the extract or pharmaceutical composition containing albiflorin is used to prevent or treat the coronavirus pneumonia by one or more of the following ways: resisting coronavirus, resisting inflammatory storm, restoring intestinal flora balance and resisting hypoxia; and further preferably, the albiflorin or the pharmaceutically acceptable salt thereof or the extract or pharmaceutical composition containing albiflorin is used to prevent or treat the coronavirus pneumonia by one or more of the following ways: inhibiting coronavirus 3CLpro protein, promoting endogenous bile acid secretion, inhibiting sphingosine kinase SphK1 and/or SphK2, inhibiting interleukin-6 or phospholipase A2/arachidonic acid (PLA2/AA) inflammatory factors, inhibiting an IDO1 inflammatory signaling pathway, regulating intestinal flora balance and promoting EPO production to resist hypoxia.

In the present invention, a molecular docking research is performed on albiflorin and four screened SARS-ConV inhibitors with high similarity, and it is found that they all act on a 3CLpro protein target. On this basis, the inventors have studied the binding mode of albiflorin and the (2019-nCoV) 3CLpro protein target by using a computer molecular docking method, and calculated the binding degree of freedom therebetween, and found that albiflorin is a high potential (2019-nCoV) 3CLpro protein inhibitor, thus albiflorin or a pharmaceutically acceptable salt thereof, or an extract or a pharmaceutical composition containing albiflorin can be prepared into a medicament or health care product for the treatment of coronavirus pneumonia (especially COVID-19) by inhibiting coronavirus (especially 2019-nCoV) 3CLpro protein.

In the present invention, upon antidepressant researches on chronic stress rats through targeted metabolomics, it is found that albiflorin or the pharmaceutically acceptable salt thereof, or the extract or pharmaceutical composition containing albiflorin can promote secretion of endogenous bile acid from the body, increase the immunity, and resist novel coronavirus (2019-nCoV).

In the present invention, upon anti-inflammatory and immunomodulatory studies in vitro and in vivo, it is found that albiflorin or the pharmaceutically acceptable salt thereof, or the extract or pharmaceutical composition containing albiflorin is an inhibitor of inflammatory factors such as interleukin-1β, interleukin-6, phospholipase A2/arachidonic acid (PLA2/AA), etc., which can inhibit sphingosine kinase to promote the synthesis of sphingosine-1-phosphate (S1P), is an inhibitor for SphK1, SphK2, IDO1 inflammatory pathways, and can prevent the “inflammatory storm” induced by influenza pneumonia, coronavirus pneumonia, especially novel coronavirus pneumonia (COVID-19) through anti-inflammatory and immune regulation.

In the present invention, upon metabolomics and 16sRNA high-throughput sequencing studies, it is found that albiflorin or the pharmaceutically acceptable salt thereof, or the extract or pharmaceutical composition containing albiflorin has a function of regulating the intestinal flora balance, and can regulate the microecological balance of the human body by increasing intestinal metabolites, such as bile acid, amino acids and vitamins and by increasing the abundance of various lactic acid bacteria in the intestinal tract, thereby preventing and treating secondary infection caused by bacterial translocation, assisting in the treatment of coronavirus pneumonia, especially novel coronavirus pneumonia (COVID-19).

In the present invention, through a hypoxia tolerance experiment, it is found that albiflorin or the pharmaceutically acceptable salt thereof, or the extract or pharmaceutical composition containing albiflorin can increase the level of erythropoietin EPO in a hypoxic state of the body, increase the oxygen carrying capacity of red blood cells, and relieve the lack of blood oxygen saturation caused by coronavirus pneumonia, especially the novel coronavirus pneumonia (COVID-19), thereby prolonging the survival time of hypoxic patients, which is not only crucial for rescuing the lives of critically ill patients with novel coronavirus pneumonia, but also provides long-term conditioning treatment for residual diseases of patients with impaired lung function and insufficient blood oxygen delivery after recovery.

Compared with the prior art, the present invention has the following advantages:

1. The present invention has the comprehensive advantages of multiple functions, such as resisting viruses and inflammations, improving intestinal flora balance and resisting hypoxia, etc., so as to synergistically prevent and treat the novel coronavirus pneumonia:

An antiviral effect can be achieved by inhibiting the novel coronavirus (2019-nCoV) 3CLpro protein and increasing bile acids in the body.

An anti-inflammatory effect can be achieved by inhibiting inflammatory factors such as interleukin-6 and phospholipase A2/arachidonic acid (PLA2/AA), etc., inhibiting SphK1 and SphK2, and inhibiting inflammatory pathways such as IDO1, etc., so as to prevent the inflammatory storm; in addition, SphK2 is an essential component for the novel coronavirus to replicate in cells, and if it is inhibited, the virus will not replicate. Therefore, albiflorin inhibits the effect of SphK2, which can not only reduce the inflammation level, but also prevent the replication of the virus, and can also play the function of protecting brain nerves.

Secondary infection caused by bacterial translocation is inhibited by improving the microecological balance of the human body.

The red blood cells can be stimulated to increase the oxygen carrying capacity and resist hypoxia by increasing the level of the erythropoietin EPO.

Therefore, albiflorin or pharmaceutically acceptable salt thereof or the extract or pharmaceutical composition containing albiflorin can be used in the treatment of early- and mid-stage novel coronavirus pneumonia (COVID-19).

2. Albiflorin or the pharmaceutically acceptable salt thereof, or the extract or pharmaceutical composition containing albiflorin of the present invention is a natural drug derived from traditional Chinese medicinal materials, is safe with few side effects and has high patient dependency, and can be not only used for the treatment of coronavirus pneumonia, especially the early- and mid-term treatment of novel coronavirus pneumonia (COVID-19), and turn treatment into prevention and treatment by inhibiting the advance of the “inflammatory storm” at early stage, but also used for the rehabilitation of patients with novel coronavirus pneumonia after recovery, helping patients reduce adverse consequences, especially helping to restore physical strength, brain vitality and glucose metabolism.

3. Albiflorin or the pharmaceutically acceptable salt thereof, or the extract or pharmaceutical composition containing albiflorin of the present invention can increase the oxygen carrying capacity of red blood cells by increasing the level of erythropoietin (EPO) which stimulate production of red blood cells, and relieve the lack of blood oxygen saturation caused by coronavirus pneumonia, especially the novel coronavirus pneumonia (COVID-19), thereby prolonging the survival time of hypoxic patients, which is crucial for rescuing the lives of critically ill patients.

4. Albiflorin or the pharmaceutically acceptable salt thereof or the extract or pharmaceutical composition containing albiflorin of the present invention has anti-depression, anti-anxiety and sleep-improving effects, and can be used to treat depression and sleep disorders co-morbid with novel coronavirus pneumonia (COVID-19).

### BRIEF DESCRIPTION OF THE DRAWINGS

FIG. 1 shows inhibitors of SARS-ConV;

FIG. 2 shows that albiflorin may treat COVID-19 by inhibiting 3CLpro protease;

FIG. 3 shows a binding mode of albiflorin in 3CLpro protease;

FIGS. 4A-4D show the binding modes of molecules 101-104 in 3CLpro protease, respectively, wherein FIG. 4A shows a result of docking of the molecule 101 with 6LU7, FIG. 4B shows a result of docking of the molecule 102 with 6LU7, FIG. 4C shows a result of docking of the molecule 103 with 6LU7, and FIG. 4D shows a result of docking of the molecule 104 with 6LU7;

FIG. 5 shows the changes of bile acid secretion in depression model animals;

FIG. 6 shows that albiflorin increases bile acid secretion by regulating intestinal flora;

FIG. 7 shows an effect of paeoniflorin on the secretion of IL-6 in the blood of acute stress model mice;

FIG. 8 shows an effect of albiflorin on the secretion of IL-6 in the blood of acute stress model mice;

FIG. 9 shows that albiflorin inhibits the increase of cPLA2 in chronic stress rats;

FIG. 10 shows that albiflorin resists inflammations by inhibiting cPLA2 in chronic stress rats;

FIG. 11 shows an effect of albiflorin on SphK1 and SphK2 detected by Western Blot;

FIG. 12 shows an effect of albiflorin on IDO1 detected by Western Blot;

FIG. 13 shows targeted metabolomics multiple comparison analysis (PLS-DA);

FIG. 14 shows gut population structure cluster analysis in high-throughput metagenomic sequencing after albiflorin administration; and

FIG. 15 shows that the disturbance of intestinal microbial metabolism in the rats of a model group is corrected in the albiflorin group (the Top 25 metabolites with significant changes after administration).

### BEST MODE FOR CARRYING OUT THE INVENTION

The present invention will be further described below in conjunction with specific examples. However, the following examples of the present invention are only used to illustrate the present invention, but not to limit the scope of the present invention.

### Example 1: Computer Molecular Docking Study of Albiflorin Inhibiting a 3CLpro Protein of 2019-nCoV

### 1.1 Experimental Method

2019-nCoV, SARS-CoV, MERS-CoV, etc. are all coronaviruses, and their processes of invading a host are the same, so the corresponding drug development strategies are also similar. The coronavirus relies on the binding of a Spike protein on the surface to an angiotensin-converting enzyme 2 (ACE2) receptor on the surface of a host cell, and then enters a recipient cell. After entering the recipient cell, viral positive-sense RNA is translated into two long peptide chains by host ribosome, and the two long peptide chains are subjected to a proteolysis process, and then cut and assembled into corresponding functional proteins. This proteolysis process is mainly completed by coronavirus main protease (3CLpro) and papain-like protease (PLpro). Coronavirus RNA polymerase (RdRp) is responsible for replicating a viral RNA genome to generate new viral individuals. Therefore, four proteins of Spike, 3CLpro, PLpro and RdRp are the key enzymes of virus invasion and reproduction, and thus become the most important therapeutic targets.

At present, the sequencing of the whole genome of 2019-nCoV has been completed. According to this gene sequence, the sequences of corresponding proteins of 2019-nCoV, SARS-CoV and other viruses can be compared to find differences, thereby guiding drug development. Compared with a Spike protein of SARS-CoV, a Spike protein of 2019-nCoV has undergone major changes in some key areas, resulting in a decline in the effectiveness of drugs targeting the Spike protein of SARS-CoV for 2019-nCoV. In contrast, the three key targets of 3CLpro, RdRp and PLpro of 2019-nCoV have more than 95% sequence similarity with those of SARS-CoV. Therefore, active compounds developed against SARS-CoV may have some therapeutic effect on COVID-19.

Albiflorin is a natural product with complex activities. The inventor believes that this compound may have an inhibitory effect on 2019-nCoV, and hopes to preliminarily prove through a method of computer-aided drug design. To achieve this goal, it is first necessary to determine which target of 2019-nCoV albiflorin is most likely to have an effect on. Among the four key proteins of 2019-nCoV, only 3CLpro has completed the analysis of protein crystals, and the other three can only be subjected to a decking study through homology models, resulting in a larger error. Therefore, the inventors use a small molecule similarity method to identify the most likely targets.

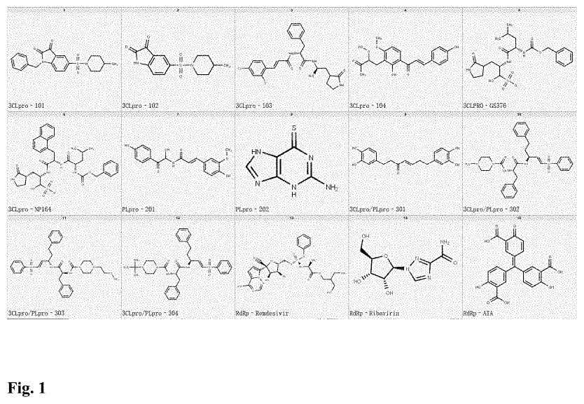

Based on the above ideas, the inventors first collect 15 active compounds developed against the three key targets of 3CLpro, RdRp and PLpro of SARS-ConV from the literatures, as shown in FIG. 1. Next, the molecular structure similarity of albiflorin and these 15 SARS-ConV inhibitors is compared using molecular fingerprints in MOE software. The molecular similarity algorithm has been applied to the activity prediction of albiflorin and other natural products for many times, and has been proved to have a good accuracy rate.

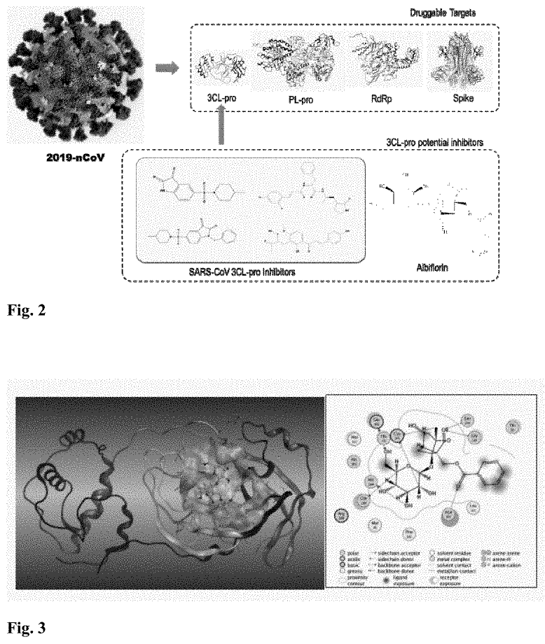

Table 1 lists the top 5 SARS-ConV inhibitors with relatively high similarity to albiflorin, and it can be found that 4 of them all act on a 3Clpro protein target. Therefore, the inventors believe that if albiflorin has an inhibitory effect on 2019-nCoV, this compound most likely works by inhibiting the 3Clpro protein target, see FIG. 2.

TABLE 1
Five SARS-ConV inhibitors with high similarity to albiflorin

| Target | Name | Similarity |
|---|---|---|
| 3CLpro | GS376 | 81% |
| 3CLpro | 103 | 80% |
| RdRp | ATA | 64% |
| 3CLpro | 101 | 50% |
| 3CLpro/PLpro | 302 | 47% |

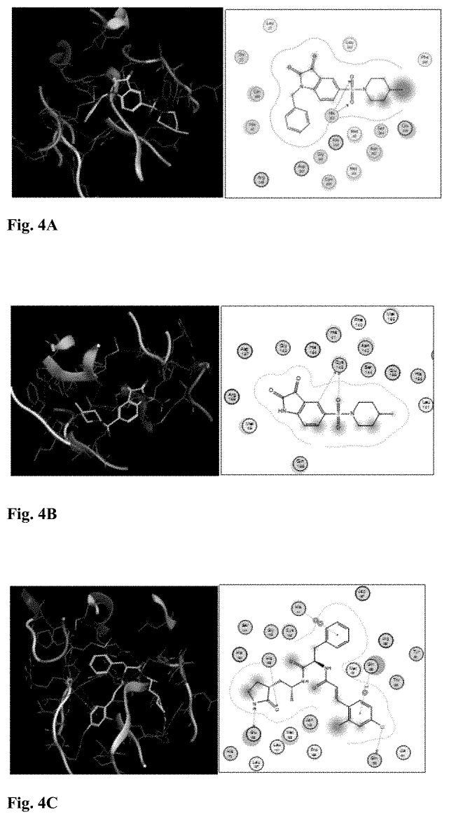

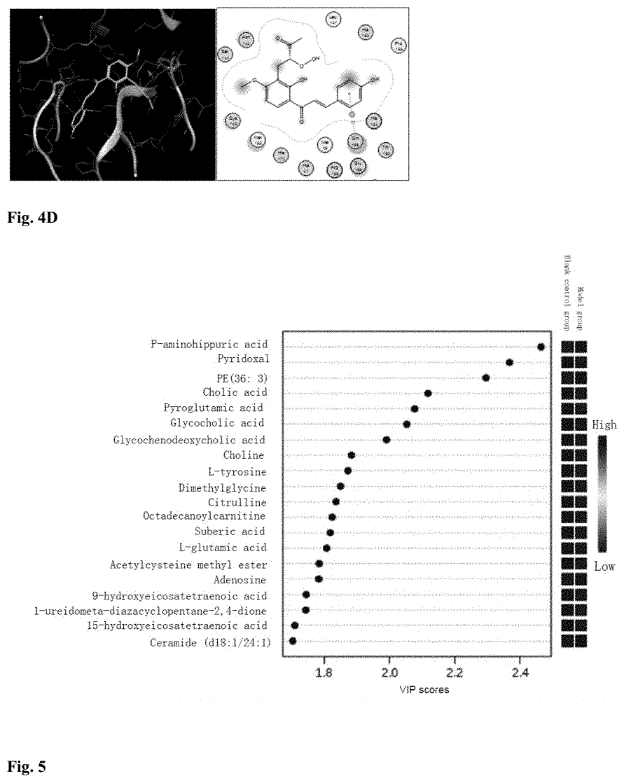

Next, the inventors study a binding mode of albiflorin and the 3CLpro protein target through molecular docking, and roughly calculate the binding free energy thereof. In order to have a comparison, the inventors use 3CLpro-101 to 104 in FIG. 1 which act on SARS-ConV, and simulate the binding mode of these four molecules with the 3CLpro protein target also by a molecular docking method and calculate the intensities, as shown in FIG. 3. A template for molecular docking is a crystal structure having a PDB number of 6LU7, which is jointly released by the Institute of Immunochemistry, ShanghaiTech University and the Shanghai Institute of Materia Medica, Chinese Academy of Sciences, as shown in FIG. 4.

### 1.2 Results

From the molecular docking results, albiflorin and four other 3CLpro protein inhibitors of SARS-ConV can bind well to the 3CLpro protein of 2019-nCoV. The binding modes are relatively similar, which proves that the docking results are more reliable.

The binding free energy of 5 molecules is between −48 and −90 kCal/mol. Since this binding free energy is calculated using a molecular mechanics method in MOE software, the error is relatively large, this binding free energy has guiding significance because it is still on the order of magnitude level, and thus can be used to illustrate that these five molecules bind to the 3CLpro protein of 2019-nCoV at relatively high strength.

Due to the large number of oxygen atoms in the molecular structure, albiflorin easily interacts variously with amino acid residues in a receptor pocket. Upon the comparison of these five molecules, it can be found that Cys145, Glu166, and Gln189 are important residues in the interaction between ligands and receptors. The results are shown in Table 2.

TABLE 2
Comparison of albiflorin and other four ligands

| | Binding | |
|---|---|---|
| | free energy | |
| Ligands | (kCal/mol) | Important residues |
| Albiflorin | −90.895 | His41, Gly143, Ser144, Cys145, |
| | | Glu166, Gln189 |
| 101 | −65.193 | His163 |
| 102 | −50.346 | Cys145 |
| 103 | −70.643 | His41, His163, Glu166, Gln189, Gln192 |
| 104 | −48.677 | Gln189 |

The research results of Example 1 can prove that albiflorin is a 3CLpro protein inhibitor of 2019-nCoV with high research potential.

### Example 2: Albiflorin Increases Endogenous Bile Acid Secretion by Regulating Intestinal Flora

### 2.1 Experimental Animals

32 healthy male SD rats, weighing 180-220 g, are randomly divided into a blank control group (Control), a model group (CUMS), a fluoxetine group (FLX), and an albiflorin group (Albiflorin), with 8 in each group. They are reared in 2 cages, adaptively fed for a week before the experiment, had free access to water and food, and are trained with 1% sucrose water.

Except for the blank control group, all groups receive randomly designed stress stimulation, and are then administrated intragastrically with fluoxetine (10 mg/kg/d) and albiflorin (7 mg/kg/d) for 7 consecutive days in terms of 1.0 mL/100 g body weight after 29 days of stress stimulation, while the corresponding stress stimulation is continued in the course of administration. All drugs are prepared into solutions or suspensions with physiological saline before use, and dissolved by ultrasonic waves.

### 2.2 Collection of Metabolomic Samples

After 7 days of administration, the administration ends, and 24 hours later, behavioral tests are performed. After the tests, the animals are anesthetized and sacrificed. The plasma, hippocampal tissues, and feces of the animals are collected separately, and preserved for later use by using appropriate methods according to the experimental requirements.

### 2.3 Extraction of Small Molecule Metabolites from Tissues and Plasma

Hippocampus: The hippocampus of the rats is accurately weighed, added with 9 times the volume of a pre-cooled extraction solution (methanol-acetonitrile-acetone-water 30:30:30:10; V/V/V/V), homogenized by ultrasonic waves, mixed well by vortexing and stood on ice for 10-15 minutes to make the extraction solution and animal tissue powder fully react. The main purpose of this step is to lyse cell walls and precipitate macromolecular substances such as proteins and DNA, etc. After centrifuging for 10 minutes at high speed and low temperature (16000 g, 4° C.), small molecule metabolites are in supernatant in a tube. 200 μl of the supernatant is taken and placed into a new centrifuge tube and dried with nitrogen for later use.

Feces: the feces of the rats are accurately weighed, added with 9 times the volume of a pre-cooled extraction solution (methanol-acetonitrile-water (42:42:16; V/V/V), homogenized by ultrasonic waves, mixed well by vortexing and stood on ice for 10-15 minutes to make the extraction solution and the feces fully react. The main purpose is to lyse cell and precipitate macromolecular substances such as proteins and DNA, etc. After centrifuging for 10 minutes at high speed and low temperature (16000 g, 4° C.), small molecule metabolites are in supernatant in the tube. 200 μl of the supernatant is taken and placed into a new centrifuge tube and dried with nitrogen for later use.

Plasma: 100 μl of plasma is taken and transferred into a 1.5 ml centrifuge tube. 400 μl of pre-cooled extraction solution (methanol-water 50:50; V/V) is added, and mixed well by vortexing. The tube is stood on ice for 10 minutes to make the extraction solution and plasma fully react, and centrifuged at high speed (16000 g, 4° C.) for 10 minutes. Small molecule metabolites are in supernatant in the tube. 200 μl of the supernatant is taken and placed into a new centrifuge tube and dried with nitrogen for later use.

### 2.4 LC-MS/MS Next-Generation Targeted Metabolomic Analysis

The metabolite extract is taken, dissolved in 100 μl mobile phase, and the main metabolites in the sample are determined by LC-MS/MS (Shimadzu LC-20AD-Qtrap 5500 tandem mass spectrometer (SCIEX, USA)). Chromatographic separation conditions: chromatographic column: aPHera amino column (150×2 mm, 4 μm, Supelco, USA), mobile phase: A: 95% ultrapure water+5% acetonitrile+20 μM ammonia water, B: 100% acetonitrile; flow rate: 0.5 ml/min, column temperature: 25° C., and injection volume: 10 μl. Elution conditions: 0-3 min, 95% B; 3-6 min, 75% B; 6-7 min, 0% B; 7-12 min, 0% B, 12-15 min, 95% B. Mass spectrometry conditions: ion source: electrospray (ESI), which adopts a fast switching mode of positive and negative particles, with a switching rate of 50 ms. Ion source temperature: 500° C., gas 1: 30 psi, gas 2: 30 psi, curtain gas: 30 psi, ion spray voltage: positive: 5500V, negative: −4500V. Scanning mode: real-time multiple reaction detection mode (Scheduled MRM). There are 625 MRM ion pairs (625 major metabolites, covering 62 major metabolic pathways of organisms).

### 2.5 Bioinformatics Data Analysis

The obtained chromatographic peak information (Wiff file) is imported into Multiquant 3.0 (SCIEX, USA), and the obtained chromatographic peaks are area-integrated and checked manually. The correctly-checked chromatographic peak areas are imported into Excel for max/min, Z-score, and missing value analysis. The analyzed data is imported into Metaboanalyst for multivariate analysis (PLS-DA, OPLS-DA and VIP analysis), metabolic pathways analysis and correlation analysis.

### 2.6 The Changes of Bile Acid Secretion in Depression Model Animals

Through VIP analysis, the differences between intestinal flora metabolism of the depression model rats and the blank control group are explored. The inventors find that the intestinal flora metabolism of depression model rats is significantly lower than that of the blank control group. Among the significant Top 20 metabolites in both groups, 16 (80%) metabolites are significantly reduced (VIP>1.5) in the depression model rat group, see FIG. 5.

From the perspective of chemical classification, these metabolites that are reduced in the intestinal flora metabolism of depression rats mainly include: amino acids and vitamins (vitamin B6 (Pyridoxal), choline, L-tyrosine, citrulline and L-glutamic acid, etc.), bile acids (cholic acid and glycocholic acid)) and nucleic acid and its derivatives.

Cholic acid is a main bile acid produced by the liver using cholesterol, which is excreted into the intestine through the enterohepatic circulation, and further metabolized into various other metabolites by microorganisms in the intestine. In addition, recent studies have reported that intestine bacteria themselves can synthesize a series of sterol bile acids from metabolites in the intestine. As hormones, these bile acids are reabsorbed into the body and play a very important role in regulating fat metabolism, energy metabolism and inflammatory responses. The bile acid content in the feces of the depression model rats is significantly reduced, indicating that the intestinal bile acid reabsorption rate is increased.

### 2.7 The Effects of Albiflorin on Bile Acid Secretion in Depression Model Animals

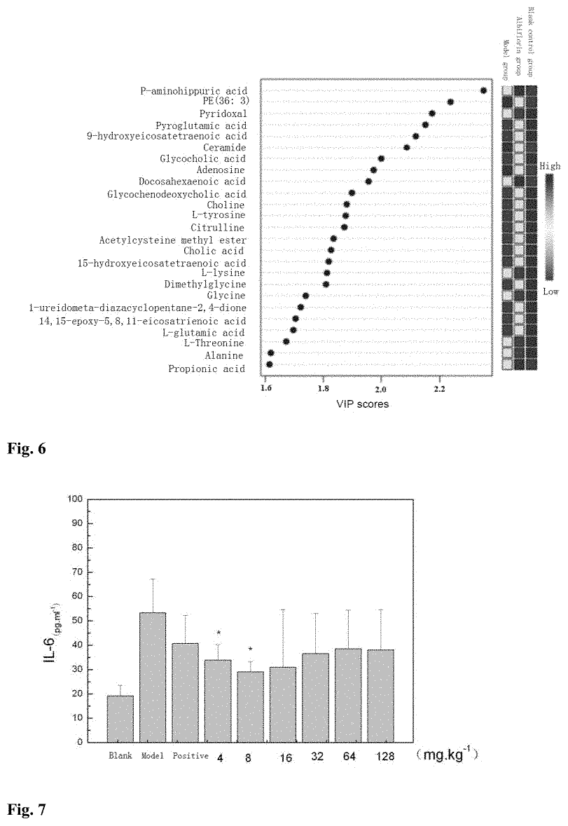

VIP analysis of the effects of albiflorin administration on the intestinal flora of depression rats shows that after administration, the overall metabolism of intestinal flora is significantly improved compared with the depression group, as shown in FIG. 6. The main manifestations are the increase of bile acid content and the increase of amino acid and vitamin content. It is indicated that the normal intestinal flora function is improved and partially recovered under the action of albiflorin.

The results of this example show that the intestinal flora metabolism of the chronic stress model rats is significantly lower than that of the blank control group, especially the reabsorption of bile acids are increased and the content of amino acids are significantly reduced.

Albiflorin administration almost completely restores the normal intestinal flora metabolism, which is manifested by reducing the reabsorption of bile acids in the intestine and increasing the content of bile acids and amino acids.

### Example 3: Study on Anti-Inflammatory and Immunomodulatory Effects of Albiflorin and Paeoniflorin

### 3.1. Materials and Methods

### 3.1.1 Experimental Animals and Main Reagents

Adult male ICR mice, weighing 18-22 g, are adaptively fed for one week, with normal diet and drinking water, and are randomly divided into 5 groups: a blank control group, a model group, a model-making fluoxetine group, a model-making administration group (with albiflorin, 6 doses of subgroups), and a model-making administration group (paeoniflorin, 6 doses of subgroups), with 5 animals in each group.

Paeoniflorin (a purity of 95.2%) is purchased from Nanjing Zelang Pharmaceutical Technology Co., Ltd., and albiflorin (a purity of 96.5%) is provided by Shanghai Eternal Biotechnology Co., Ltd. Albiflorin and paeoniflorin are formulated into aqueous solutions within 24 hours prior to administration. Dosages of adminstration (paeoniflorin, albiflorin) are 4 mg/kg, 8 mg/kg, 16 mg/kg, 32 mg/kg, 64 mg/kg, 128 mg/kg, respectively. All animals are housed in clean iron rearing cages with free access to water and food. A rearing room keeps good sound insulation conditions, a temperature of 18-24° C., a humidity of 50%-55%, and accepts 12 h light illumination every day. Mode of administration: administration is performed after one week of adaptive feeding, intragastric administration for 2 days, once a day, 0.5 ml each time, and experiments are started 2 hours after the second intragastric administration.

IL-6 ELISA Detection Kit, Invitrogen (Biosource), USA.

Multifunctional Microplate Analyzer, FlexStation 3, Molecular Devices, USA.

Milli-Q Ultrapure Water System, Millipore Corporation, USA.

### 3.1.2 Establishment of Acute Stress Model in Mice

An acute stress animal model is established by restraint braking in combination with hot and cold stimulation. The experiment is carried out between 9:00 and 15:00, and the mice in the experimental groups are put into a 50 ml plastic centrifuge tubes with ventilation at the bottom, and fixed to make them immobilized. The restrained mice are placed in a refrigerator (4° C.) for 30 min, then returned to room temperature for 10 min, placed in a ventilated oven (45° C.) for 10 min, and then kept in a restrained state for 10 min at room temperature.

### 3.1.3 Sampling

Blood is quickly collected from the femoral artery of the mouse after the above-mentioned model-making and anticoagulated with heparin sodium, and plasma is separated. After the animal is killed by decapitation, the whole brain is taken on ice, and the left hemisphere brain region is taken under the condition of ensuring that all the hypothalamus are obtained. The collected brain tissue samples are quickly put into liquid nitrogen for storage and reserved for the analysis of monoamine neurotransmitters and their metabolites. The separated plasma is labeled with groups and then stored in a −20° C. refrigerator for analysis of corticosterone and IL-6 content.

### 3.1.4 Determination of IL-6 Content in Plasma of Acute Stress Model Mice

The plasma samples stored in the −20° C. refrigerator are returned to room temperature and centrifuged at 12,000 rpm/min for 5 min, and the supernatant obtained by centrifugation is used to determine the expression level of IL-6 in the blood of acutely stressed mice by ELISA. The assay of IL-6 is performed according to the instructions of the kit.

### 3.2 Results

The effects of paeoniflorin and albiflorin on the content of IL-6 in the blood of acute stress model mice are as follows: after acute restraint of mice, immune cells such as peripheral lymphocytes and phagocytes in their plasma increase, the concentrations of some inflammatory cytokines such as IL-1β, IL-2, IFN-γ, TNF-α and IL-6 etc. increase, and the presence of high-concentration inflammatory cytokines existing for a long time will affect the own immune systems and induce depression under certain conditions.

In this experiment, an acute stress animal model is used to investigate the changes of IL-6 levels in mice under acute stress conditions. FIG. 7 shows that paeoniflorin has a certain inhibitory effect on the secretion of IL-6 in the blood of acute stress mice in the dose range of 4, 8, 16, 32, 64, and 128 mg/kg, and has the most significant inhibitory effect (P<0.05) at the dose of 4 mg/kg and 8 mg/kg; and FIG. 8 shows that albiflorin has a certain inhibitory effect on the secretion of IL-6 in acute stress mice in a low dose range, and has the most significant inhibitory effect (P<0.01) at the dose of 8 mg/kg, but with the increase of the administration dose, its inhibitory effect is not obvious, and the secretion of IL-6 is promoted obviously.

### Example 4 Anti-Inflammatory Study of Albiflorin by Inhibiting cPLA2

### 4.1 Albiflorin (ALB) Inhibits the Increase of cPLA2 in Chronic Stress Rats

The inventors have established a chronic unpredictable mild stress rat model (CUMS), wherein rats under test are divided into a blank control group (Ctrl-sal), a chronic stress model group (CUMS-sal), a fluoxetine administration group (CUMS-flx) and an albiflorin administration group (CUMS-Alb).

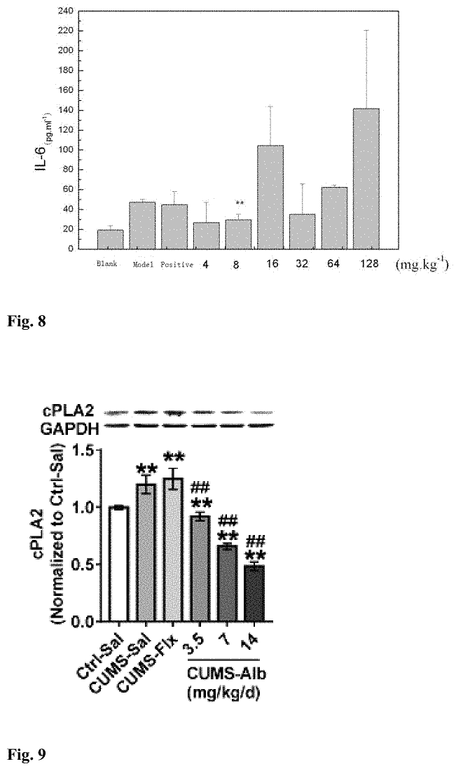

Phospholipase A2 (cPLA2) is significantly increased in the hippocampus of chronic stress (CUMS) model rats. After 7 days of albiflorin administration (3.5 mg/day, 7 mg/day, 14 mg/day), phospholipase A2 (cPLA2) in the hippocampus of rats is significantly decreased (P<0.01), showing a significant dose dependency, see FIG. 9.

### 4.2 Anti-Inflammation of Albiflorin (ALB) by Inhibiting cPLA2 in Chronic Stress Rats

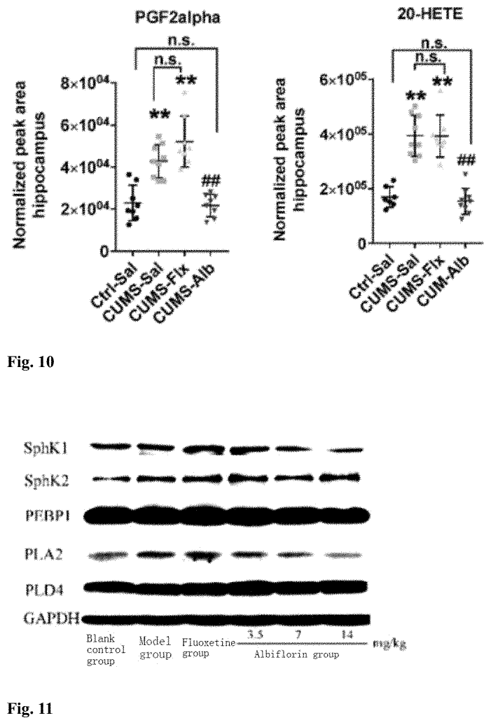

It is found in experiments that chronic stress (CUMS) induces a significant increase in the expression of cPLA2 in the hippocampus of model rats, resulting in an increase in inflammatory mediators PGF2α and 20-HETE. Albiflorin (ALB) administered for 7 days (7 mg/kg) can inhibit the increase of cPLA2, reduce the content of inflammatory mediators PGF2α and 20-HETE, reduce hippocampal neuroinflammation, and restore the homeostasis of membrane lipids, see FIG. 10.

### Example 5: Inhibition of Albiflorin on Hippocampal Sphingosine Kinase in Chronic Stress Rats

Sphingosine kinases (SphK1, SphK2) are significantly increased in the hippocampus of chronic stress (CUMS) model rats. After 7 days of albiflorin administration (7 mg/day, 14 mg/day), sphingosine kinases (SphK1, SphK2) in the hippocampus of rats are significantly decreased (P<0.01). The results are shown in the following table.

Inhibitory effects of albiflorin on SphK1 and SphK2 in hippocampus of chronic stress rats

| Group | | SphK1 | SphK2 |
|---|---|---|---|
| Blank control group | | 1.0 ± 0.007** | 1.0 ± 0.007** |
| Model group | | 1.1 ± 0.007 | 1.8 ± 0.005 |
| Fluoxetine group | | 1.5 ± 0.009## | 2.1 ± 0.016## |
| Albiflorin group | 3.5 | 1.2 ± 0.009## | 2.2 ± 0.016## |
| (mg/kg) | 7 | 0.7 ± 0.004** | 1.6 ± 0.010** |
| | 14 | 0.5 ± 0.001** | 2.1 ± 0.015## |

##means an increase compared with the model group, p < 0.01;
*means a decrease compared with the model group, p < 0.05;
**means a decrease compared with the model group, p < 0.01.

The effects of albiflorin on SphK1 and SphK2 are detected by Western Blot, see FIG. 11.

Conclusion: sphingosine kinases (SphK1, SphK2) serve as main rate-limiting enzymes in the synthesis of sphingosine-1-hydrochloride in cells. As can be seen from FIG. 11, the sphingosine kinases are significantly increased in the hippocampus of CUMS model rats, suggesting that chronic stress inhibits the synthesis of sphingosine-1-hydrochloride. Medium- and high-dose administration of albiflorin can significantly reduce the content of sphingosine kinases (SphK1, SphK2) in rat hippocampus, indicating that albiflorin can increase the content of sphingosine-1-hydrochloride in rat hippocampus, thereby promoting the proliferation and survival of hippocampal cells.

Albiflorin is a sphingosine kinase 2 (SphK2) inhibitor. SphK2 is a component necessary for the replication of the novel coronavirus in cells. If it is inhibited, the virus will not replicate. Therefore, the inhibitory effects of albiflorin on SphK2 can not only reduce the inflammation level, but also prevent the viral replication, and also play the functions of protecting brain nerves.

### Example 6: Inhibition of Albiflorin on Overexpression of IDO1 in the Hippocampus of Chronic Stress Rats

The secretion of IDO1 is significantly increased in the hippocampus of chronic stress (CUMS) rats. After 7 days of albiflorin administration (7 mg/day, 14 mg/day), the secretion of IDO1 in the hippocampus of rats is significantly inhibited (P<0.01). The results are shown in the following table.

Inhibitory effects of albiflorin on IDO1 in hippocampus of chronic stress rats

| Group | | IDO1 |
|---|---|---|
| Blank control group | | 1.0 ± 0.010** |
| Model group | | 1.0 ± 0.011# |
| Fluoxetine | | |
| Albiflorin group | 3.5 g | 1.0 ± 0.010## |
| (mg/kg) | 7 | 0.8 ± 0.007** |
| | 14 | 0.7 ± 0.007** |

#means an increase compared with the model group, p < 0.05;
##means an increase compared with the model group, p < 0.01; and
**means a decrease compared with the model group, p < 0.01.

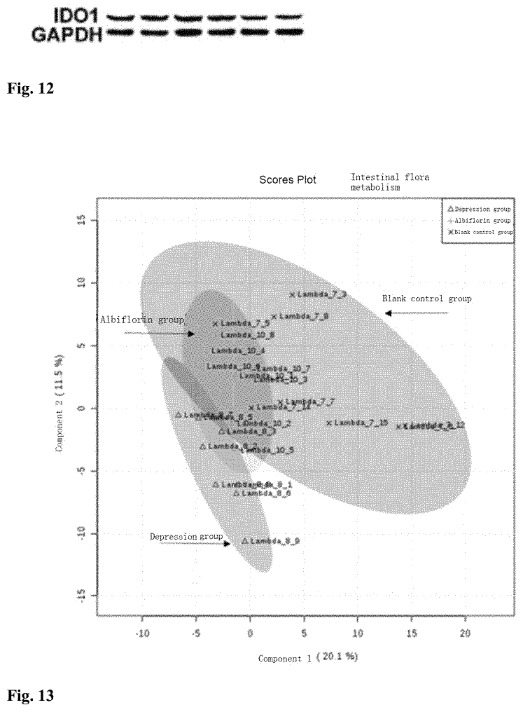

The effects of albiflorin on IDO1 is detected by Western Blot, see FIG. 12.

Conclusion: albiflorin has a significant inhibitory effect on overexpression of IDO1 in hippocampus of chronic stress (CUMS) rats.

### Example 7: Study on Albiflorin for Restoring Intestinal Flora Balance in CUMS Rats

The inventors establish the chronic unpredictable mild stress rat model (CUMS). Using a new generation of targeted metabolomics methods, the metabolic function of the intestinal flora of stress rats is studied. PLS-DA multivariate analysis shows that there is a significant difference between the intestinal flora metabolism of rats in the depression model group (depression group) and the blank control group, see FIG. 13.

The inventors further provide treatments for the stress model rats with albiflorin for 7 consecutive days, and each treatment dose is 7 mg/kg/d. Seven days later, the applicant reassesses the functions and structures of intestinal flora of the rats by using metabolomics and 16sDNA high-throughput metagenomic sequencing technology. It is manifested through multiple comparison analysis (PLS-DA) of metabolomic data that after albiflorin administration, the intestinal flora of rats is metabolized and moved toward the blank control group, which is almost completely overlapped with the blank control group, indicating that albiflorin helps to restore normal metabolism of intestinal flora in stress rats, as shown in FIG. 13. This is consistent with the results of the flora structure analysis.

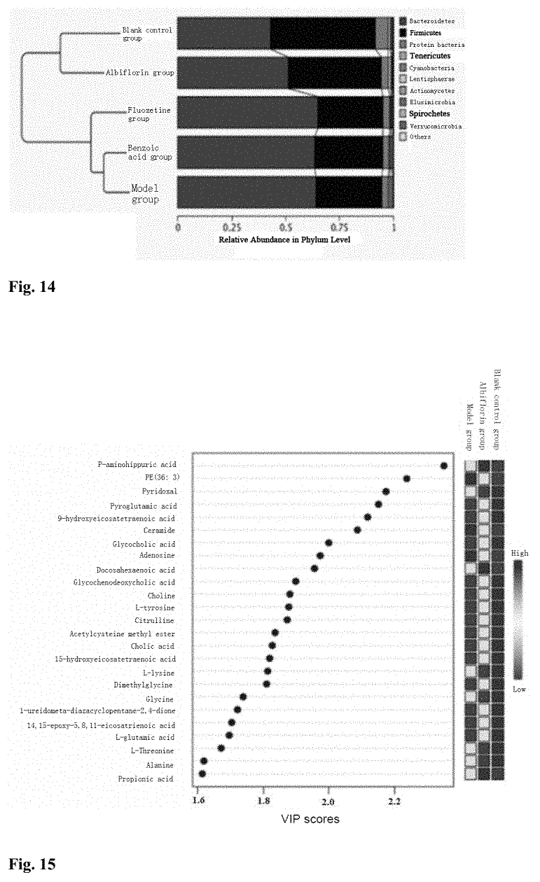

Albiflorin administration group (Alb) has similar flora structures with the blank control group (Control), both of which are clustered together without any statistical difference (P>0.05), and has a significant increase in the content of beneficial bacteria Firmicutes, especially lactic acid bacteria compared with the stress rat model group, see FIG. 14.

VIP analysis shows that after 7 days of albiflorin administration, the overall metabolism of intestinal flora is significantly improved compared with the depression group, which is mainly manifested in the increase in the content of bile acids and the content of amino acids and vitamins, see FIG. 15.

1.-6. (canceled)

7. A method for preventing or treating coronavirus pneumonia, comprising: administering a prophylactically or therapeutically effective amount of albiflorin or a pharmaceutically acceptable salt thereof or an extract or a pharmaceutical composition containing albiflorin to a subject in need thereof.

8. A method for treating prolonged symptoms of novel coronavirus pneumonia, performing rehabilitation conditioning after recovery of novel coronavirus pneumonia, or alleviating a possible sequela of novel coronavirus pneumonia, comprising: administering a prophylactically or therapeutically effective amount of albiflorin or a pharmaceutically acceptable salt thereof or an extract or a pharmaceutical composition containing albiflorin to a subject in need thereof, wherein the prolonged symptoms of the novel coronavirus pneumonia are pain, palpitation, asthma, consciousness disorder, or chronic fatigue; and/or the sequela is depression, anxiety, sleep disorder, pain, palpitation, asthma, intestinal function disorder or chronic fatigue syndrome.

9. The method according to claim 7, wherein the extract containing albiflorin is total glucosides of Paeonia lactiflora Pall and/or a Paeonia lactiflora Pall extract, and/or the pharmaceutical composition is a Chinese herbaceous peony and licorice preparation.

10. A medicament, a health care product or a nutrition regulator for preventing or treating coronavirus pneumonia, comprising albiflorin or a pharmaceutically acceptable salt thereof or an extract or a pharmaceutical composition containing albiflorin.

11. The method according to claim 7, wherein the coronavirus pneumonia is novel coronavirus pneumonia.

12. The method according to claim 7, wherein the albiflorin or the pharmaceutically acceptable salt thereof or the extract or pharmaceutical composition containing albiflorin is used to prevent or treat the coronavirus pneumonia by one or more of the following ways: resisting coronavirus, resisting inflammatory storm, restoring intestinal flora balance and resisting hypoxia.

13. The method according to claim 7, wherein the albiflorin or the pharmaceutically acceptable salt thereof or the extract or pharmaceutical composition containing albiflorin is used to prevent or treat the coronavirus pneumonia by one or more of the following ways: inhibiting coronavirus 3CLpro protein, promoting endogenous bile acid secretion, inhibiting sphingosine kinase SphK1 and/or SphK2, inhibiting interleukin-6 or phospholipase A2/arachidonic acid inflammatory factors, inhibiting an IDO1 inflammatory signaling pathway, regulating intestinal flora balance and promoting EPO production to resist hypoxia.

14. The method according to claim 8, wherein the extract containing albiflorin is total glucosides of Paeonia lactiflora Pall and/or a Paeonia lactiflora Pall extract, and/or the pharmaceutical composition is a Chinese herbaceous peony and licorice preparation.

15. The medicament, health care product or nutrition regulator according to claim 10, wherein the extract containing albiflorin is total glucosides of Paeonia lactiflora Pall and/or a Paeonia lactiflora Pall extract, and/or the pharmaceutical composition is a Chinese herbaceous peony and licorice preparation.

16. The medicament, health care product or nutrition regulator according to claim 10, wherein the medicament, health care product or nutrition regulator is selected from capsules, tablets, dropping pills, preparations for nasal administration or injections.

17. The medicament, health care product or nutrition regulator according to claim 10, wherein the albiflorin or the pharmaceutically acceptable salt thereof or the extract or pharmaceutical composition containing albiflorin is used to prevent or treat the coronavirus pneumonia by one or more of the following ways: resisting coronavirus, resisting inflammatory storm, restoring intestinal flora balance and resisting hypoxia.

18. The medicament, health care product or nutrition regulator according to claim 10, wherein the albiflorin or the pharmaceutically acceptable salt thereof or the extract or pharmaceutical composition containing albiflorin is used to prevent or treat the coronavirus pneumonia by one or more of the following ways: inhibiting coronavirus 3CLpro protein, promoting endogenous bile acid secretion, inhibiting sphingosine kinase SphK1 and/or SphK2, inhibiting interleukin-6 or phospholipase A2/arachidonic acid inflammatory factors, inhibiting an IDO1 inflammatory signaling pathway, regulating intestinal flora balance and promoting EPO production to resist hypoxia.
